(19) United States
(12) Patent Application Publication
(10) Pub. No.: US 2026/0116863 A1
(43) Pub. Date: Apr. 30, 2026

(54) SYNTHESIS METHOD OF GLYCIDYL NEODECANOATE

(71) Applicant: Hebei Raysen Chemical Co., Ltd., Handan (CN)

(72) Inventors: Xiaolong LI, Handan (CN); Ronggang ZHANG, Handan (CN); Shulong HAO, Handan (CN)

(73) Assignee: Hebei Raysen Chemical Co., Ltd., Handan (CN)

(21) Appl. No.: 19/365,062

(22) Filed: Oct. 21, 2025

(30) Foreign Application Priority Data
Oct. 26, 2024 (CN) 202411504703.8

(51) Int. Cl. C07D 301/30

(52) U.S. Cl. CPC C07D 301/30

(57) ABSTRACT

The present invention relates to the field of organic synthesis technology and proposes a synthesis method for glycidyl neodecanoate, comprising the following steps: S1. polymerization reaction of neodecanoic acid and epichlorohydrin as raw materials under the action of a catalyst to obtain an intermediate; S2. Substitution reaction of the intermediate and liquid alkali by reversal addition and treatment to obtain glycidyl neodecanoate. The above technical solution resolves problems of pipeline blockage and low utilization of liquid alkali in the two-step synthesis of diglycidyl neodecanoate in related technologies.

### RELATED APPLICATIONS

The present application claims the benefit of priority of Chinese Patent Application No. 202411504703.8, filed Oct. 26, 2024, and entitled “A SYNTHESIS METHOD OF GLYCIDYL NEODECANOATE,” the entire content of which is incorporated herein by reference.

### TECHNICAL FIELD

The present invention relates to the field of organic synthesis technology, specifically relates to a method for synthesizing diglycidyl neodecanoate.

### BACKGROUND

Glycidyl neodecanoate is widely used in the field of coatings and can be used as a raw material or additive. The synthesis methods of glycidyl neodecanoate include one-step method and two step method. For one-step method, neodecanoic acid reacts directly with epichlorohydrin in the presence of a catalyst to generate glycidyl neodecanoate. This method is simple to operate, but the reaction conditions are relatively harsh for requiring high temperature and high pressure and the product yield is low. For two step method, firstly a ring opening reaction is performed between neodecanoic acid and epichlorohydrin under the action of a catalyst to generate neodecanoic acid chlorohydrin ester, and then a ring closing reaction is performed to obtain glycidyl neodecanoate. This method has relatively mild reaction conditions and high product yield, but involves multiple reaction steps and complex operations.

Among them, in the closed-loop reaction of the two-step method where a one-time addition of alkali is used, it is prone to pipeline blockage and low utilization rate of liquid alkali. Therefore, a new method for synthesizing glycidyl neodecanoate is needed to avoid pipeline blockage, improve the utilization rate of liquid alkali, and enhance the quality and market competitiveness of the product when preparing glycidyl neodecanoate by the two-step method.

### SUMMARY

The present invention proposes a method for synthesizing glycidyl neodecanoate, which solves the problems of pipeline blockage and low utilization of liquid alkali in the two-step synthesis of glycidyl neodecanoate in related technologies.

The technical solution of the present invention is as follows:

The present invention proposes a method for synthesizing diglycidyl neodecanoate, comprising the following steps:
S1. polymerization reaction of neodecanoic acid and epichlorohydrin as raw materials under the action of catalyst to obtain intermediates;
S2. substitution reaction of the intermediate and liquid alkali by reversal addition and treatment to obtain glycidyl neodecanoate.

As a further embodiment, the reversal addition is achieved by reversal alkali addition device.

As a further embodiment, the post-treatment comprises standing and dehydration treatment in sequence.

As a further embodiment, the mass ratio of the neodecanoic acid, the epichlorohydrin and the liquid alkali is 1.5˜2:1:0.2˜0.3.

As a further embodiment, the temperature of the substitution reaction is 65-75° C. and the time of the substitution reaction is 2-3 hours.

As a further embodiment, the polymerization reaction temperature is 65-75° C. and the polymerization reaction time is 4-5 hours.

As a further embodiment, the addition of epichlorohydrin is carried out dropwise at a rate of 0.08˜0.11 kg/s.

As a further embodiment, the catalyst comprises one or two of tetramethylammonium neodecanoate and tetramethylammonium chloride.

As a further embodiment, the amount of catalyst added is 0.1% to 0.2% of the mass of neodecanoic acid.

As a further embodiment, the catalyst is a nickel platinum catalyst and the preparation method of the nickel platinum catalyst comprises the following steps:
A1. mixing platinum salt, nickel salt and water and adjusting the pH to alkaline and obtain a precipitate;
A2. washing the precipitate until neutral, drying and calcining the mixture to obtain a nickel platinum catalyst.

As a further embodiment, the mass ratio of platinum salt to nickel salt is 5:1 to 3.

In the present invention, nickel platinum catalyst is used for the synthesis of glycidyl neodecanoate, especially when the mass ratio of platinum salt to nickel salt is 5:1˜3, which further improves the purity and yield of glycidyl neodecanoate.

As a further embodiment, the platinum salt comprises one or two of chloroplatinic acid and potassium chloroplatinate; the nickel salt includes one or both of nickel nitrate and nickel acetate.

The working principle and beneficial effects of the present invention are:

In the present invention, the reverse alkali addition method is used in the closed-loop reaction of the two-step method, which can reduce pipeline blockage, improve the utilization rate of liquid alkali, and synthesize new decanoic acid glycidyl ester with high purity and yield.

### DETAILED DESCRIPTION

The following technical solutions in the embodiments of the present invention will be clearly and completely described in conjunction with the embodiments of the present invention. Obviously, the described embodiments are only a part of the embodiments of the present invention. Based on the embodiments of the present invention, all other embodiments obtained by ordinary skilled persons in the art without creative labor are within the scope of protection of the present invention.

### Embodiment 1

The synthesis method of diglycidyl neodecanoate includes the following steps:

Aggregation reaction: new decanoic acid is added to the reaction vessel through a new decanoic acid metering tank, with a one-time addition amount of 2291.276 kg. The material in the reaction vessel is heated to 70° C. by using a thermal oil furnace, and then epichlorohydrin is added dropwise through an epichlorohydrin metering tank for 4 hours. During the dropwise addition process, the pH value of the solution in the reaction vessel is constantly monitored. When the pH is less than 1, the dropwise addition is stopped with a dropwise acceleration rate of 0.087 kg/s, and the one-time addition amount is 1251.543 kg. After the dropwise addition is completed, the catalyst tetramethylammonium neodecanoate and tetramethylammonium chloride are added to the reaction vessel in a mass ratio of 1:1, with a one-time addition amount of 3.03 kg. Finally, the thermal oil furnace is used for heating, and the temperature inside the reaction vessel is controlled. The mixture is incubate at 70° C. for 4 hours to obtain the intermediate;

Substitution reaction: raising the intermediate to 70° C. and using a reverse alkaline hydrolysis device to add liquid alkali with an alkaline hydrolysis period of 2 hours. When the chlorine content in the oil layer is less than 100 ppm, standing the mixture and dehydrating it to obtain glycidyl neodecanoate. Among them, the mass ratio of neodecanoic acid to epichlorohydrin to and liquid alkali is 1.831:1:0.246.

The final yield of glycidyl neodecanoate was 92.0%, with a purity of 94.1% and a liquid alkali utilization rate of 97.0%.

### Embodiment 2

The synthesis method of diglycidyl neodecanoate includes the following steps:

Aggregation reaction: new decanoic acid is added to the reaction vessel through a new decanoic acid metering tank, with a one-time addition amount of 2291.276 kg. The material in the reaction vessel is heated to 65° C. by using a thermal oil furnace, and then epichlorohydrin is added dropwise through an epichlorohydrin metering tank for 4 hours. During the dropwise addition process, the pH value of the solution in the reaction vessel is monitored at real time. When the pH is less than 1, the dropwise addition is stopped, with a dropwise acceleration rate of 0.080 kg/s and a one-time addition amount of 1145.638 kg. After the dropwise addition is completed, tetramethylammonium chloride is added to the reaction vessel, with a one-time addition amount of 2.29 kg. Finally, the reaction vessel is heated using a thermal oil furnace, and the temperature is controlled at 65° C. for 5 hours to obtain the intermediate;

Substitution reaction: raising the intermediate to 65° C. and use a reverse alkaline hydrolysis device to add liquid alkali. The alkaline hydrolysis time is 3 hours. When the chlorine content in the oil layer is less than 100 ppm, standing the mixture and dehydrating it to obtain glycidyl neodecanoate. Among them, the mass ratio of neodecanoic acid to epichlorohydrin to liquid alkali is 2:1:0.3.

The final yield of glycidyl neodecanoate was 92.6%, with a purity of 94.9% and a liquid alkali utilization rate of 97.2%.

### Embodiment 3

The synthesis method of diglycidyl neodecanoate includes the following steps:

Aggregation reaction: New decanoic acid is added to the reaction vessel through a new decanoic acid metering tank, with a one-time addition amount of 2291.276 kg. The material in the reaction vessel is heated to 75° C. by using a thermal oil furnace, and then epichlorohydrin is added dropwise through an epichlorohydrin metering tank for 4 hours. During the dropwise addition process, the pH value of the solution in the reaction vessel is monitored at any time. When the pH is less than 1, the dropwise addition is stopped, with a dropwise acceleration rate of 0.106 kg/s and a one-time addition amount of 1527.517 kg. After the dropwise addition is completed, tetramethylammonium chloride is added to the reaction vessel, with a one-time addition amount of 4.58 kg. Finally, the reaction vessel is heated using a thermal oil furnace, and the temperature is controlled at 75° C. for 4 hours to obtain the intermediate;

Substitution reaction: raising the intermediate to 75° C. and use a reverse alkaline hydrolysis device to add liquid alkali. The alkaline hydrolysis time is 2 hours. When the chlorine content in the oil layer is less than 100 ppm, standing the mixture and dehydrating it to obtain glycidyl neodecanoate. Among them, the mass ratio of neodecanoic acid, epichlorohydrin, and liquid alkali is 1.5:1:0.2.

The final yield of glycidyl neodecanoate was 92.2%, with a purity of 95.1% and a liquid alkali utilization rate of 97.6%.

### Embodiment 4

The difference between this embodiment and embodiment 1 is only that the catalyst is replaced with a nickel molybdenum catalyst. The preparation method of the nickel molybdenum catalyst includes the following steps:
A1. mixing molybdenum nitrate, nickel nitrate and water in a mass ratio of 5:4 and adjusting the pH to 9 to obtain a precipitate;
A2. washing the precipitate until neutral, drying and calcining the mixture to obtain a nickel molybdenum catalyst.

The yield and purity of the resulting diglycidyl neodecanoate are 93.0% and 97.6%, respectively.

### Embodiment 5

The difference between this embodiment and embodiment 1 is only that the catalyst is replaced with a nickel platinum catalyst. The preparation method of the nickel platinum catalyst includes the following steps:
A1. mixing platinum nitrate, nickel nitrate in water with a mass ratio of 5:4 and adjust the pH to 9 to obtain a precipitate;
A2. washing the precipitate until neutral, drying and calcining the mixture to obtain a nickel platinum catalyst.

The final yield of the newly synthesized diglycidyl decanoate is 94.7%, with a purity of 97.5%.

### Embodiment 6

The difference between this embodiment and embodiment 1 is only that the catalyst is replaced with a nickel platinum catalyst. The preparation method of the nickel platinum catalyst includes the following steps:
A1. mixing platinum nitrate, nickel nitrate in water in a mass ratio of 10:1 and adjust the pH to 9 to obtain a precipitate;
A2. washing the precipitate until neutral, drying and calcining the mixture to obtain a nickel platinum catalyst.

The final yield of the newly synthesized diglycidyl decanoate is 94.1%, with a purity of 98.0%.

### Embodiment 7

The difference between this embodiment and embodiment 1 is only that the catalyst is replaced with a nickel platinum catalyst. The preparation method of the nickel platinum catalyst includes the following steps:
A1. mixing platinum nitrate, nickel nitrate in water with a mass ratio of 5:1 and adjust the pH to 9 to obtain a precipitate;
A2. washing the precipitate until neutral, drying and calcining the mixture to obtain a nickel platinum catalyst.

The final yield of the newly synthesized diglycidyl decanoate is 96.8%, with a purity of 98.4%.

### Embodiment 8

The difference between this embodiment and embodiment 1 is only that the catalyst is replaced with a nickel platinum catalyst. The preparation method of the nickel platinum catalyst includes the following steps:
A1. mixing platinum nitrate, nickel nitrate in water with a mass ratio of 5:3 and adjust the pH to 9 to obtain a precipitate;
A2. washing the precipitate until neutral, drying and calcining the mixture to obtain a nickel platinum catalyst.

The final yield of the newly synthesized diglycidyl decanoate is 97.0%, with a purity of 98.9%.

### Embodiment 9

The difference between this embodiment and embodiment 1 is only that the catalyst is replaced with a nickel platinum catalyst. The preparation method of the nickel platinum catalyst includes the following steps:
A1. mix platinum nitrate, nickel acetate, and water in a mass ratio of 5:3 and adjust the pH to 10 to obtain a precipitate;
A2. washing the precipitate until neutral, drying and calcining the mixture to obtain a nickel platinum catalyst.

The final yield of the newly synthesized diglycidyl decanoate is 97.8%, with a purity of 98.6%.

### Comparison Embodiment 1

The only difference between this comparative embodiment and Embodiment 1 is the substitution reaction in the synthesis method of diglycidyl neodecanoate: the intermediate is heated to 70° C. and then added with liquid alkali, and the alkaline hydrolysis time is 2 hours. When the chlorine content in the oil layer is less than 100 ppm, it is allowed to stand and dehydrated to obtain diglycidyl neodecanoate.

The final yield of glycidyl neodecanoate was 85.0%, with a purity of 89.6% and a liquid alkali utilization rate of 90.5%.

Compared with Comparison embodiment 1, the liquid alkali utilization rate of the new decanoic acid glycidyl ester prepared in Embodiments 1-9 is higher, while the yield and purity are also higher, indicating that the reverse alkali addition method is used in the two-step closed-loop reaction, and the utilization rate of liquid alkali is high. The synthesized new decanoic acid glycidyl ester also has the characteristics of high purity and yield.

Compared with Embodiments 1 and 4, the yield and purity of the glycidyl neodecanoate obtained in Embodiments 5 to 9 are higher, indicating that the use of nickel platinum catalyst in the synthesis of the glycidyl neodecanoate further improved its purity and yield.

Compared with Embodiments 5-6, the yield and purity of the glycidyl neodecanoate obtained in Embodiments 7-9 are higher, indicating that the use of nickel platinum catalyst in the synthesis of glycidyl neodecanoate, especially when the mass ratio of platinum salt to nickel salt is 5:1-3, further improves the purity and yield of glycidyl neodecanoate.

The above are only the preferred embodiments of the present invention and are not intended to limit the present invention. Any modifications, equivalent substitutions, improvements, etc. made within the spirit and principles of the present invention should be included in the scope of protection of the present invention.

What is claimed is:

1. A method for synthesizing diglycidyl neodecanoate, comprising the following steps:
S1. polymerization reaction of neodecanoic acid and epichlorohydrin as raw materials under the action of catalyst to obtain intermediates;
S2. substitution reaction of the intermediate and liquid alkali and treatment to obtain glycidyl neodecanoate,
The liquid alkali is added by reversal addition.

2. The method for synthesizing diglycidyl neodecanoate according to claim 1, characterized in that the post-treatment comprises standing and dehydration treatment in sequence.

3. The method for synthesizing glycidyl neodecanoate according to claim 1, characterized in that the mass ratio of the neodecanoic acid to the epichlorohydrin to the liquid alkali is 1.5˜2:1:0.2˜0.3.

4. The method for synthesizing glycidyl neodecanoate according to claim 1, characterized in that the temperature of the substitution reaction is 65-75° C. and the time of the substitution reaction is 2-3 hours.

5. The method for synthesizing glycidyl neodecanoate according to claim 1, characterized in that the polymerization reaction temperature is 65-75° C. and the polymerization reaction time is 4-5 hours.

6. The method for synthesizing glycidyl neodecanoate according to claim 1, characterized in that the addition of epichlorohydrin is carried out dropwise at a rate of 0.08˜0.11 kg/s.

7. The method for synthesizing glycidyl neodecanoate according to claim 1, characterized in that the catalyst comprises one or two of tetramethylammonium neodecanoate and tetramethylammonium chloride.

8. The method for synthesizing glycidyl neodecanoate according to claim 1, characterized in that the amount of catalyst added is 0.1% to 0.2% of the mass of neodecanoic acid.

9. The method for synthesizing glycidyl neodecanoate according to claim 7, characterized in that the catalyst is a nickel platinum catalyst and the preparation method of the nickel platinum catalyst comprises the following steps:
A1. mixing platinum salt, nickel salt and water and adjusting the pH to alkaline and obtain a precipitate;
A2. washing the precipitate until neutral, drying and calcining the mixture to obtain a nickel platinum catalyst.

10. The method for synthesizing glycidyl neodecanoate according to claim 9, characterized in that the mass ratio of platinum salt to nickel salt is 5:1 to 3.
